(19) United States
(12) Patent Application Publication
(10) Pub. No.: US 2004/0060127 A1
(43) Pub. Date: Apr. 1, 2004

(54) Composition for dyeing keratin fibers, comprising at least one para-phenylenediamine derivative comprising a pyrrolidyl group substituted with a silyl radical

(76) Inventors: Stephane Sabelle, Paris (FR); Laure Ramos, Bourg Lareine (FR); Madeleine Leduc, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/603,831

(22) Filed: Jun. 26, 2003

Related U.S. Application Data
(60) Provisional application No. 60/408,899, filed on Sep. 9, 2002.

(30) Foreign Application Priority Data
Jun. 26, 2002 (FR) 02 07939

(51) Int. Cl. A61K 7/13

(52) U.S. Cl. 8/405

(57) ABSTRACT

Disclosed herein are: a composition for dyeing keratin fibers, for example, human keratin fibres such as hair, comprising at least one para-phenylenediamine derivative comprising a pyrrolidyl group substituted with a silyl radical; a process for dyeing keratin fibers using this composition; and novel para-phenylenediamine derivatives comprising a pyrrolidyl group substituted with a silyl, radical.

This application claims benefit of U.S. Provisional Application No. 60/408,899, filed Sep. 9, 2002.

Disclosed herein is a composition for dyeing keratin fibers, for example, human keratin fibers such as hair, comprising at least one para-phenylenediamine, derivative comprising a pyrrolidyl group substituted with a silyl radical.

It is known practice to dye keratin fibers, for example, human hair, with dye compositions containing oxidation dye precursors, such as ortho- or para-phenylenediamines, ortho- or para-aminophenols, heterocyclic compounds such as diaminopyrazole derivatives, pyrazolo[1,5-a]pyrimidine derivatives, pyrimidine derivatives, pyridine derivatives, 5,6-dihydroxyindole derivatives and 5,6-dihydroxyindoline derivatives, which are generally known as oxidation bases. Oxidation dye precursors, or oxidation bases, are colorless or weakly colored compounds which, when combined with oxidizing products, can give rise to colored compounds and dyes by a process of oxidative condensation.

It is also known that the shades obtained with these oxidation bases may be varied by combining them with couplers or coloration modifiers, and the couplers or coloration modifiers can be chosen, for example, from meta-phenylenediamines, meta-aminophenols, meta-hydroxyphenols and certain heterocyclic compounds, for example, pyrazolo[1,5-b]-1,2,4-triazole derivatives, pyrazolo[3,2-c]-1,2,4-triazole derivatives, pyrazolo[1,5-a]pyrimidine derivatives, pyridine derivatives, 5-pyrazolone derivatives, indoline derivatives and indole derivatives.

The variety of molecules used as oxidation bases and couplers can allow a wide range of colors to be obtained.

The “permanent” coloration obtained using these oxidation dyes should usually satisfy a certain number of requirements. For example, it should have no toxicological drawbacks, it should produce shades in the desired intensity, and should show good resistance to external agents, such as light, bad weather, washing, permanent-waving, perspiration and rubbing.

The dyes should also be able to cover grey hair and should be as unselective as possible, i.e., they should produce the smallest possible differences in coloration along the same, length of a keratin fiber, which may in fact be differently sensitized (i.e., damaged) between its end and its root. They should also show good chemical stability in the formulations and should have a good toxicological profile.

In the field of hair dyeing, para-phenylenediamine and para-tolylenediamine are oxidation bases that are widely used. They can produce varied shades with oxidation couplers.

However, there is a need to discover novel oxidation bases with a better toxicological profile than para-phenylenediamine and para-tolylenediamine, while at the same time being able to give the hair excellent properties in terms of color intensity, variety of shades, color uniformity and resistance to external agents.

It is known to use para-phenylenediamine derivatives substituted with a pyrrolidine group as oxidation bases for coloring keratin fibers. For example, U.S. Pat. No. 5,851,237 describes the use of 1-(4-aminophenyl)pyrrolidine derivatives optionally substituted on the benzene nucleus as replacements for para-phenylenediamine.

U.S. Pat. No. 5,993,491 proposes the use of N-(4-aminophenyl)-2-hydroxymethylpyrrolidine derivatives optionally substituted on the benzene nucleus and on the pyrrolidine heterocycle in position 4 with a hydroxyl radical, as replacements for para-phenylenediamine.

Patent application JP 11-158 048 proposes compositions containing at least one compound chosen from 4-aminoaniline derivatives optionally substituted on the benzene nucleus and one of the nitrogen atoms of which is included in a 5- to 7-membered carbon-based ring.

However, it is clearly established that these compounds cannot give hair a colorations equivalent in quality to that obtained with para-phenylenediamine or with para-tolylenediamine due to a lack of intensity and uniformity of the color.

There is thus a real need to discover novel oxidation bases that can simultaneously have a good toxicological profile and properties such that the compositions containing them can give the hair at least one of excellent properties in terms of color intensity, variety of shades, color uniformity and resistance to various external attacking factors to which the hair may be subjected.

The present inventors have thus developed novel dye compositions for dyeing keratin fibers that do not have at least one of the drawbacks of the oxidation bases of the prior art and do not degrade the keratin fibers, while at the same time are capable of producing intense colorations in varied shades, which can be relatively unselective and particularly resistant and can show a good toxicological profile.

Disclosed herein is a composition for dyeing keratin fibers, for example, human keratin fibers such as the hair, comprising, in a medium suitable for dyeing, at least one oxidation base chosen from para-phenylenediamine derivatives substituted with a pyrrolidyl group of formula (I), and the addition salts thereof

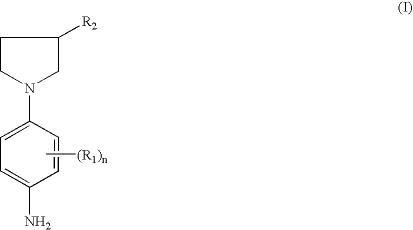

wherein

n is chosen from 0 to 4, provided that when n is greater than or equal to 2, then the radicals R₁ may be-identical or different,

R₁ is chosen from halogen atoms; saturated and unsaturated, aliphatic and alicyclic C₁-C₆ hydrocarbon-based chains, wherein at least one carbon atom of the hydrocarbon-based chains may be replaced with at least one entity chosen from oxygen, nitrogen, silicon and sulphur atoms and an SO₂ group; and at least one of the hydrocarbon-based chains may be substituted with at least one entity chosen from halogen atoms and hydroxyl, amino and mono- and di(C₁-C₄)alkylamino radicals; provided that the radical R₁ does not comprise a peroxide bond, or a diazo, nitro or nitroso radical;

R₂ is chosen from

—SiR₃R₄R₅ radicals

linear and branched C₁-C₈ alkyl radicals, which may be unsaturated, substituted with at least one radical chosen from —SiR₃R₄R₅ radicals; wherein at least one carbon atom of the alkyl radicals is possibly replaced with at least one atom chosen from oxygen and nitrogen atoms and at least one of the alkyl radicals is possibly substituted with at least one group chosen from hydroxyl, amino, (C₁-C₆)alkylamino and di(C₁-C₆)alkylamino groups, and

triarylsilanyl, triarylsulanylalkyl, triarylsilanylalkoxy, triarylsilanylalkylamine and bis(triarylsilanylalkyl)amine radicals,

wherein R₃, R₄ and R₅, which may be identical or different, are each chosen from tri(C₁-C₄)alkylsilyl radicals; a triphenylsilyl radical; a phenyl radical; and C₁-C₆ alkyl radicals which may be substituted with at least one group chosen from tri(C₁-C₆)alkylsilyl, hydroxyl, amino, (C₁-C₆)alkylamino and di(C₁-C₆)alkylamino groups.

Further disclosed herein, are novel para-phenylenediamine derivatives comprising a pyrrolidyl group substituted with a silyl radical.

Even further disclosed herein is the use of the para-phenylenediamine derivatives of formula (I) to dye keratin fibers and the, process for dyeing keratin fibers, for example, human keratin fibers such as hair, using the composition disclosed herein.

The composition disclosed herein can, for example, produce a chromatic, powerful, relatively unselective and resistant coloration of keratin fibers.

As disclosed herein, an: aliphatic hydrocarbon-based chain is chosen from linear and branched chains that may comprise at least one unsaturation chosen from unsaturations of the alkene type and the alkyne type. An alicyclic hydrocarbon-based chain is a branched chain comprising a cyclic structure, which may comprise at least one unsaturation chosen from unsaturations of the alkene, type and the alkyne type, but which does not contain an aromatic ring structure.

For example, when a carbon atom of the hydrocarbon-based chain is replaced with an entity, Y, chosen from oxygen, sulphur, nitrogen and silicon atoms and a SO₂ group, a unit —CH₂—Y—CH₂— is obtained.

By way of example, R₁ may be chosen from a chlorine atom and methyl, ethyl, isopropyl, vinyl, allyl, methoxymethyl, hydroxyethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl, 1-amino-2-hydroxyethyl, 1,2-diaminoethyl, methoxy, ethoxy, allyloxy and 2-hydroxyethyloxy radicals.

In formula (I), n is, for example, equal-to 0 or 1.

In one embodiment, R₁ is chosen from halogen atoms, for example, bromine and chlorine; C₁-C₄ alkyl radicals, C₁-C₄ hydroxyalkyl radicals, C₁-C₄ aminoalkyl radicals, C₁-C₄ alkoxy radicals and C₁-C₄ hydroxyalkoxy radicals. By way of example, R₁ is chosen from methyl, isopropyl, hydroxymethyl, 2-hydroxyethyl, 1,2-dihydroxyethyl, methoxy, isopropyloxy and 2-hydroxyethoxy radicals,

The radical R₂ is chosen, for example, from trialkylsilanyl, trialkylsilanylalkyl, trialkylsilanylalkoxy, trialkylsilanylalkylamine, bis(trialkylsilanylalkyl)amine, triarylsilanyl, triarylsilanylalkyl, triarylsilanylalkoxy, triarylsilanylalkylamine and bis(triarylsilanylalkyl)amine radicals. In one embodiment, R₂ is chosen from trialkylsilanyl, trialkylsilanylalkyl, trialkylsilanylalkoxy, trialkylsilanylalkylamine and bis(trialkylsilanylalkyl)amine radicals.

The radicals R₃, R₄ and R₅, which may be identical or different, are each chosen, for example, from alkyl radicals chosen, for example, from methyl, ethyl, propyl, isopropyl, butyl, isobutyl, tert-butyl, pentyl, hexyl and cyclohexyl radicals, and phenyl and tolyl radicals.

The compounds of formula (I) may be in the form of acid salts with at least one acid chosen from strong mineral acids, for example, HCl, HBr and H₂SO₄, and organic acids, for example, acetic acid, lactic acid, tartaric acid, citric acid and succinic acid.

Examples of the para-phenylenediamine derivatives of formula (I) that may be mentioned include:

| | | | |
|---|---|---|---|
| 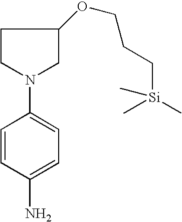 | 4-[3-(3-Tri- methylsilanyl- propoxy)-1- pyrrolidyl]- phenylamine | 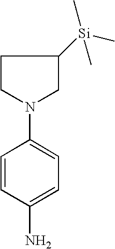 | 4-(3-Tri- methylsilanyl-1- pyrrolidyl)- phenylamine |
| 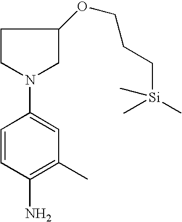 | 2-Methyl-4-[3-(3- trimethyl- silanylpropoxy)-1- pyrrolidyl]- phenylamine | 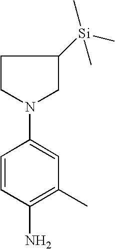 | 2-Methyl-4-(3- trimethylsilanyl-1- pyrrolidyl)- phenylamine |
| 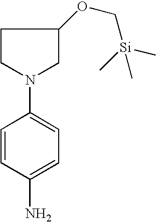 | 4-(3-Tri- methylsilanyl- methoxy-1- pyrrolidyl)- phenylamine | 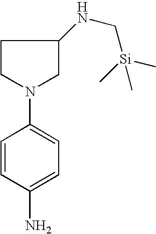 | [1-(4-Amino- phenyl)-3- pyrrolidyl]trimethyl- silanylmethylamine |
| 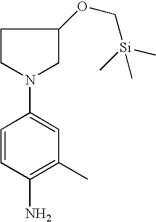 | 2-Methyl-4-(3- trimethylsilanyl- methoxy-1- pyrrolidyl)- phenylamine | 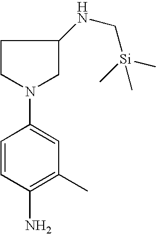 | [1-(4-Amino-3- methylphenyl)-3- pyrrolidyl]- trimethylsilan- ylmethylamine |
| 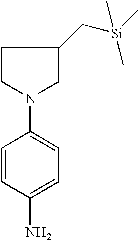 | 4-(3-Trimethyl- silanylmethyl-1- pyrrolidyl)- phenylamine | 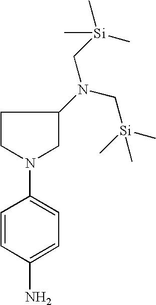 | [1-(4-Amino- phenyl)-3- pyrrolidyl]bis- (trimethylsilanyl- methyl)amine |
| 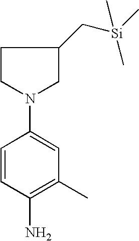 | 2-Methyl-4-(3- trimethylsilanyl- methyl-1- pyrrolidyl)- phenylamine | 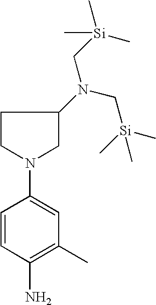 | [1-(4-Amino-3- methylphenyl)-3- pyrrolidyl]- bis(trimethyl- silanylmethyl)- amine |
| 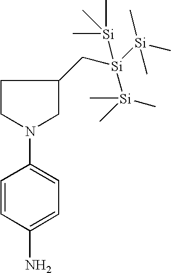 | 4-(3-Tri(trimethyl- silanyl)silanylmethyl- 1-pyrrolidyl)- phenylamine | 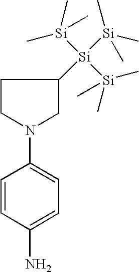 | 4-(3-Tri(tri- methylsilanyl)- silanyl-1- pyrrolidyl)- phenylamine |
| 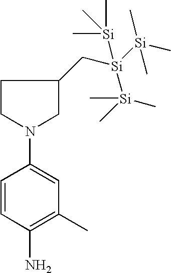 | 2-Methyl-4-(3- tri(trimethyl- silanyl)silanyl- methyl-1- pyrrolidyl)- phenylamine | 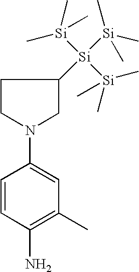 | 2-Methyl-4-(3- (tri(trimethyl- silanyl)silanyl-1- pyrrolidyl) phenylamine |
| 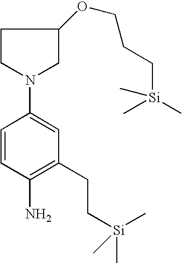 | 2-(2-Trimethyl- silanylethyl)-4-[3- (3-trimethylsilanyl- propoxy)-1- pyrrolidyl]- phenylamine | 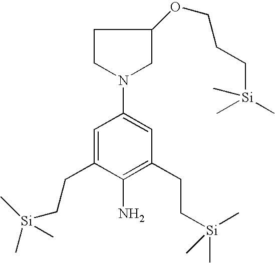 | 2,6-Bis(2-tri- methylsilanylethyl)- 4-[3-(- trimethylsilanyl- propoxy)-1- pyrrolidyl]- phenylamine |
| 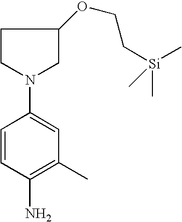 | 2-Methyl-4-[3-(3- trimethyl- silanylethyloxy)-1- pyrrolidyl]- phenylamine | 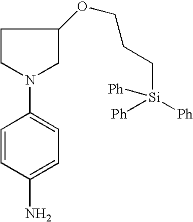 | 4-[3-(3-Tri- phenylsilanyl- propoxy)-1- pyrrolidyl]- phenylamine |
| 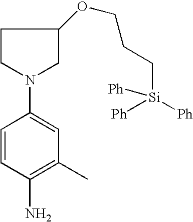 | 2-Methyl-4-[3-(3- triphenyl- silanylpropoxy)-1- pyrrolidyl]- phenylamine | 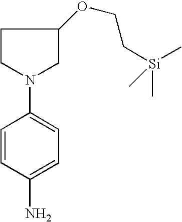 | 4-[3-(3-Tri- methylsilanyl- ethyloxy)-1- pyrrolidyl]- phenylamine |

Among these compounds, the following compounds can be used:

4-[3-(3-Trimethylsilanylpropoxy)-1-pyrrolidyl]phenylamine,

4-(3-Trimethylsilanyl-1-pyrrolidyl)phenylamine,

4-(3-(Trimethylsilanylmethoxy-1-pyrrolidyl)phenylamine,

[1-(4-Aminophenyl)-3-pyrrolidyl]trimethylsilanylmethylamine,

4-(3-Tri methylsilanyl methyl-1-pyrrolidyl)phenylamine,

[1-(4-Aminophenyl)-3-pyrrolidyl]bistrimethylsilanylmethylamine,

2-(2-Tri methylsilanylethyl)-4-[3-(3-trimethylsilanylpropoxy)-1-pyrrolidyl]phenylamine,

4-[3-(3-Trimethylsilanylethyloxy)-1-pyrrolidyl]phenylamine, and

4-[3-(3-Triphenylsilanylpropoxy)-1-pyrrolidyl]phenylamine.

Formula (I) contains a chiral center at the carbon atom to which R₂ is attached. It is to be understood that formula (I) comprises enantiomers and diastereomers and mixtures thereof including racemic mixtures.

The at least one oxidation base disclosed herein is present in an amount ranging, for example, from 0.001% to 10%, such as from 0.005% to 6%, by weight relative to the total weight of the dye composition.

The dye composition disclosed herein may comprise at least one coupler chosen from couplers conventionally used for dyeing keratin fibers. Among these couplers, mention may be made, for example, of meta-phenylenediamines, meta-aminophenols, meta-diphenols, naphthalene-based couplers and heterocyclic couplers, and the addition salts thereof.

For example, 2-methyl-5-aminophenol, 5-N-(P-hydroxyethyl)amino-2-methylphenol, 6-chloro-2-methyl-5-aminophenol, 3-aminophenol, 1,3-dihydroxybenzene, 1,3-dihydroxy-2′-methylbenzene, 4-chloro-1,3-dihydroxybenzene, 2,4-diamino-1-(β-hydroxyethyloxy)benzene, 2-amino-4-(β-hydroxyethylamino)-1 methoxybenzene, 1′,3-diaminobenzene, 1,3-bis(2,4-diaminophenoxy)propane, 3-ureidoaniline, 3-ureido-1-dimethylaminobenzene, sesambl, 1-p-hydroxyethylamino-3, 4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene and the acid addition salts thereof can be used.

In the composition disclosed herein, the at least one coupler is present in an amount ranging, for example, from 0.001% to 10%, such as from 0.005% to 6%, by weight relative to the total weight of the dye composition.

The composition disclosed herein may also comprise at least one additional oxidation base chosen from oxidation bases conventionally used in oxidation dyeing. By way of example, the at least one additional oxidation base is chosen from para-phenylenediamines other than those described above, bis(phenyl)alkylenediamines, para-aminophenols, bis-para-aminophenols, ortho-aminophenols and heterocyclic bases and the addition salts thereof.

The para-phenylenediamines which can be used are chosen, for example, from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-par-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(P-hydroxyethyl)-para-phenylenediamine, N-(β, γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the acid addition salts thereof.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, phenylenedimine 2-chloro-para-phenylenediamine and 2-β-acetylaminoethyloxy-para-phenylenediamine, and the acid addition salts thereof can, for example, be used.

The bis(phenyl)alkylenediamines which can be used are chosen, for, example, from N,N′-bis(β-hydroxyethyl)-N,N′-bis(4′-aminophenyl-1,3-diaminopropanol, N,N′-bis(N-hydroxyethyl)-N,N-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2, 5-diaminophenoxy)-3,6-dioxaoctane, and the acid addition salts thereof.

The para-aminophenols which can be used are chosen, for example, from para-aminophenol, 4-amino-3-methylphenol, 4-amino-3-fluorophenol, 4-amino-3-hydroxymethylphenol, 4-amino-2-methyl phenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the acid addition salts thereof.

The ortho-aminophenols which can be used are chosen, for example, from 2-aminophenol, 2-amino-5-methylphenol, 2-amino-6-methylphenol and 5-acetamido-2-aminophenol, and the acid addition salts thereof.

The heterocyclic bases which can be used are chosen, for example, from pyridine derivatives, pyrimidine derivatives and pyrazole derivatives.

The pyridine derivatives which may be used are chosen, for example, from the compounds described, for example, in patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the acid addition salts thereof.

Other pyridine oxidation bases that can be used are chosen, for example, from the 3-aminopyrazolo[1,5-a]pyridine oxidation bases and the addition salts thereof described, for example, in patent application FR 2 801 308. By way of example, mention may be made of pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyridirie-2-carboxylic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine-3,4-diamino'pyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-aminopyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]pyrid-7-ol; and also the addition salts thereof.

The pyrimidine derivatives which can be used are chosen, for example, from the compounds described, for example, in patents DE 2 359 399; JP 88-169 571; JP 05-63124; EP 0 770 375 and patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048 and among which mention may be made, for example, of pyrazolo[1,5-a]pyrimidine-3,7-diamine; 2;5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-7(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine and 3-amino-5-methyl-7-imidazblylpropylaminopyrazolo[1,5-a]-pyrimidine, and the acid addition salts thereof and the tautomeric forms thereof, when a tautomeric equilibrium exists.

Among the pyrazole derivatives which may be used are, for example, the compounds described in, for example, patents DE 3 843 892 and DE 4 133 957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methyl pyrazole, 4,5-diamino-1-ethyl-3-(4′-methoxyphenyl)pyrazole, 4, 5-diamino-1-ethyl-3-hydroxymethylpyrazole, 4, 5-diamino-3-hydroxymethyl-1-methylpyrazole, 4, 5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3, 5-diamino-4-(β-hydroxyethyl)amino-1-methylpyrazole, and the acid addition salts thereof.

The at least one additional oxidation base disclosed herein is present in an amount ranging, for example, from 0.001% to 10%, such as from 0.005% to 6%, by weight relative to the total weight of the dye composition.

As disclosed herein, the addition salts of the oxidation bases and of the couplers that may be used herein are chosen, for example, from the acid addition salts, such as the hydrochlorides, hydrobromides, sulphates, citrates, succinates, tartrates, lactates, tosylates, benzenesulphonates, phosphates and acetates, and the base addition salts, such as sodium hydroxide, potassium hydroxide, ammonia, amines and alkanolamines.

The dye composition disclosed herein may further comprise at least one direct dye that may be chosen, for example, from nitrobenzene dyes, azo direct dyes and methine direct dyes. The at least one direct dye can be of nonionic, anionic or cationic nature.

The medium suitable for dyeing, also known as the dye support, comprises water or a mixture of water and at least one organic solvent to dissolve the compounds that would hot be sufficiently soluble in water. The at least one organic solvent may be chosen, for example, from C₁-C₄ lower alkanols, such as ethanol and isopropanol; polyols and polyol ethers such as 2-butoxyethanol, propylene glycol, propylene glycol monomethyl ether, diethylene glycol monoethyl ether and mononmethyl ether, as well as aromatic alcohols such as benzyl alcohol and phenoxyethanol.

The at least one organic solvent is, for example, present in an amount ranging from 1% to 40% by weight relative to the total weight of the dye composition, and further for example, ranging from 5% to 30% by weight relative to the total weight of the dye composition.

The dye composition disclosed herein can also comprise at least one adjuvant chosen from adjuvants conventionally used in compositions for dyeing the hair, such as anionic, cationic, nonionic, amphoteric and zwitterionic surfactants and mixtures thereof, anionic, cationic, nonionic, amphoteric and zwitterionic polymers and mixtures thereof, inorganic and organic thickeners, such as anionic, cationic, nonionic and amphoteric associative polymeric thickeners, antioxidants, penetration agents, sequestering agents, fragrances, buffers, dispersing agents, conditioners such as silicones, which may or may not be volatile or modified, film-forming agents, ceramides, preserving agents and opacifiers.

The at least one adjuvant is-present in an amount ranging, for example, from 0.01% to 20% by weight relative to the total weight of the dye composition.

Needless to say, a person skilled in the art will take care to select this or these optional additional compounds such that the advantageous properties intrinsically associated with the oxidation dye composition in accordance with the invention are not, or are not substantially, adversely affected by the addition(s) envisaged.

The pH of the dye composition disclosed herein is, for example, ranging from 3 to 12, such as from 5 to 11. It may be adjusted to the desired value using at least one agent chosen from acidifying and basifying agents usually used in the dyeing of keratin fibers, or alternatively using at least one standard buffer system.

The acidifying agents that may be used, for example, are chosen from inorganic and organic acids such as hydrochloric acid, orthophosphoric acid, sulphuric acid, carboxylic acids such as acetic acid, tartaric acid, citric acid and lactic acid, and sulphonic acids.

The basifying agents which can be used, for example, are chosen from aqueous ammonia, alkaline carbonates, alkanolamines such as mono-, di- and triethanolamine and derivatives thereof, sodium hydroxide, potassium hydroxide and the compounds of formula (II) below:

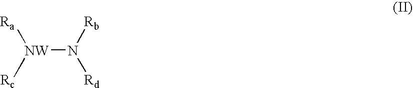

wherein W is a propylene residue which is unsubstituted or substituted with at least one entity chosen from a hydroxyl group and C₁-C₄ alkyl radicals; Rₐ, R_{b}, R_{c} and R_{d}, which may be identical or different, are each chosen from a hydrogen atom, C₁-C₄ alkyl radicals and C₁-C₄ hydroxyalkyl radicals.

The dye composition disclosed herein may be in various forms, such as in the form of liquids, creams or gels, or in any other form that is suitable for dyeing keratin fibers, such as human hair.

The process disclosed herein is a process comprising applying to the keratin fibers the composition as defined above, in the presence of at least one oxidizing agent for a time that is sufficient to develop the desired coloration. The color may be developed at acidic, neutral or alkaline pH. The at least one oxidizing agent may be mixed with the composition disclosed herein just at the time of use, or it may be used starting with an oxidizing composition comprising it, which is applied simultaneously or sequentially to the composition disclosed herein.

In one embodiment, the composition disclosed herein is mixed, for example, at the time of use, with a composition comprising, in a medium suitable for dyeing, at least one oxidizing agent, wherein the at least one oxidizing agent is present in an amount that is sufficient to develop a coloration. The mixture obtained is then applied to the keratin fibers. After an action time ranging from 3 to 50 minutes, such as from 5 to 30 minutes, the keratin fibers are rinsed, washed with shampoo, rinsed again and then dried.

The at least one oxidizing agent is chosen from oxidizing agents conventionally used for the oxidation dyeing of keratin fibers, chosen, for example, from hydrogen peroxide, urea peroxide, alkali metal bromates, persalts such as perborates and persulphates, peracids and oxidase enzymes, among which mention may be made of peroxidases, 2-electron oxidoreductases such as uricases, and 4-electron oxygenases, for example, laccases. In one embodiment, hydrogen peroxide is used.

The oxidizing composition may also comprise at least one adjuvant chosen from adjuvants conventionally used in compositions for dyeing the hair and as defined above.

The pH of the oxidizing composition comprising the at least one oxidizing agent is such that, after mixing with the dye composition, the pH of the resulting composition applied to the keratin fibers ranges, for example, from 3 to 12 and further for example, from 5 to. 11. It may be adjusted to the desired value by using at least one agent chosen from acidifying and basifying agents usually used in the dyeing of keratin fibers and as defined above.

The ready-to-use composition that is finally applied to the keratin fibers may be in various forms, such as in the form of liquids, creams or gels or any other form that is suitable for dyeing keratin fibres, for example, human hair.

Further disclosed herein is a multi-compartment dyeing device or “kit”, comprising a first compartment comprising the dye composition defined above and a second compartment comprising at least one oxidizing agent. This device may be equipped with a means for applying the desired mixture to the hair, such as the devices described in French Patent No. 2 586 913.

Using this device, it is possible to dye keratin fibers using a process that comprises mixing a dye composition comprising at least one oxidation base of formula (I) with at least one oxidizing agent, and applying the mixture obtained to the keratin fibers for a time that is sufficient to develop the desired coloration.

Even further disclosed herein are the para-phenylenediamine derivatives of formula (I) as defined above, with the exception of 5-amino-2-((3R)-3-t-butyl-dimethylsilyloxy-1-pyrrolidyl)fluorobenzene.

The compounds of formula (I) disclosed herein may be readily obtained according to methods that are well known in the prior art.

The examples that follow serve to illustrate the invention without, however, being limiting in nature.

### EXAMPLES

### Example 1

### Synthesis of 4-[3-(3-trimethylsilanylpropoxy)-1-pyrrolidyl]phenylamine Dihydrochloride (3)

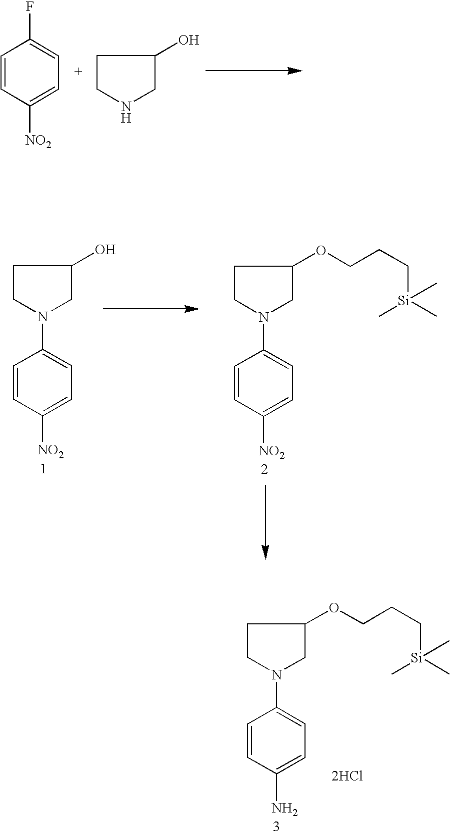

Synthesis of 1-(4-nitrophenyl)-3-pyrrolidinol (1)

2 g of 1-fluoro-4-nitrobenzene (0.0155 mol), 1.3 g of sodium hydrogen carbonate (0.0155 mol) and 15 ml of a dioxane/water mixture (8/2) were introduced into a three-necked flask. 1.35 g of racemic 3-pyrrolidinol (0.0155 mol) were quickly added to this mixture. The heterogeneous mixture was heated at reflux (87° C.) for 10 hours. The reaction mixture was then poured into ice-cold water, a yellow precipitate was obtained, which was filtered off and rinsed with water. After drying under vacuum in the presence of P₂O₅, 2.95 g of a yellow solid were obtained (97% yield).

¹H NMR (DMSO-d₆, 200 MHz, ppm): 8.04 (d, J=, 9 Hz, 2H); 6.58 (d, J=9 Hz, 2H); 5.06 (d, J=3.6 Hz, 1H); 4.41 (m, 1H); 3.45 (m, 3H); 3.20 (m, 1H); 2.04 (m, 2H).

Synthesis of N-(4-nitrophenyl)-3-(3-trimethylsilanylpropoxy)pyrrolidine (2)

0.504 g (0.02 mmol) of sodium hydride (60% in oil) was stirred in 9 ml of dimethylformamide. 2.08 g (0.01 mol) of N-(4-nitrophenyl)-3-hydroxypyrrolidine (1) were added to the reaction medium at room temperature. After stirring for 30 minutes, 1.3 g (0.0085 mol) of chloropropyltrimethylsilane were added at room temperature and the reaction medium was then heated at 80° C. for 5 hours 30 minutes. The reaction mixture was poured into water and the expected product was extracted with dichloromethane. The organic phases were evaporated under reduced pressure. The oily residue thus obtained was purified by chromatography on silica gel. 0.6 g of N-(4-nitrophenyl)-3-(3-trimethylsilanylpropoxy)pyrrolidine (2) was obtained in the form of a yellow powder. Yield=18%.

Melting point=68° C.

¹H NMR (400 MHz DMSO) ppm 8.05 (d, 2H), 6.63 (d, 2H), 4.21 (m, 1H), 3.46 (m, 6H), 2.09 (m, 2H), 1.47 (s, 2H), 0.45 (m, 2H), 0.04 (bs, 9H)

ESI+: m/z=323 (MH+), 345.(MH+22)

Synthesis of 4-[3-(3-trimethylsilanylpropoxy)-1-pyrrolidyl]phenylamine Dihydrochloride (3)

6 g of zinc powder and 0.6 g of ammonium chloride were refluxed in 15 ml of ethanol. 0.43 g (0.00134 mol) of the above derivative 2 was added slowly to the reaction mixture. Refluxing was continued for 1 hour. The mixture was allowed to cool to room temperature. After filtering off the zinc, 0.3 g of 4-[3-(3-trimethylsilanylpropoxy)-1-pyrrolidyl]-phenylamine dihydrochloride (3) was isolated in the form of the hydrochloride. Yield=61%.

¹H NMR (400 MHz, DMSO) ppm 9.95 (bs, 2H), 7.18 (d, 2H), 6.58 (d, 2H), 4.17 (m, 1H), 3.31 (m, 6H), 2.06 (m, 2H), 1.47 (m, 2H), 0.46 (m, 2H), 0.03 (s, 9H)

ESI+: m/z=292 (M+).

Examples of Dyeing in Alkaline Medium

| Examples | 1 | 2 | 3 | 4 |
|---|---|---|---|---|
| 4-[3-(3-Trimethylsilanyl- | 10⁻³ mol | 10⁻³ mol | 10⁻³ mol | 10⁻³ mol |
| propoxy)-1-pyrrolidyl] | | | | |
| phenylamine (base) | | | | |
| 2-(2,4-Diaminophenoxy) | 10⁻³ mol | — | — | — |
| ethanol dihydrochloride | | | | |
| (coupler) | | | | |
| 3-Amino-2-chloro-6- | — | 10⁻³ mol | — | — |
| methylphenol | | | | |
| hydrochloride | | | | |
| (coupler) | | | | |
| 3,6-Dimethyl-1H- | — | — | 10⁻³ mol | — |
| pyrazolo[5,1-c][1,2,4] | | | | |
| triazole (coupler) | | | | |
| 2-Amino-3-pyridinol | — | — | — | 10⁻³ mol |
| (coupler) | | | | |
| Dye support (1) | (*) | (*) | (*) | (*) |
| Demineralized water qs | 100 g | 100 g | 100 g | 100 g |

(*) Dye support (1) pH 9.5

| | |
|---|---|
| 96° ethyl alcohol | 20.8 g |
| Sodium metabisulphite as an aqueous 35% | 0.23 g A.M. |
| solution | |
| Pentasodium salt of diethylenetriaminepenta- | 0.48 g A.M. |
| acetic acid, as an aqueous 40% solution | |
| C₈-C₁₀ alkyl polyglucoside as an aqueous | 3.6 g A.M. |
| 60% solution | |
| Benzyl alcohol | 2.0 g |
| Polyethylene glycol containing 8 units of | 3.0 g |
| ethylene oxide | |
| NH₄Cl | 4.32 g |
| Aqueous ammonia containing 20% NH₃ | 2.94 g |

“A.M.” means active material.

At the time of use, each composition was mixed with an equal weight of 20-volumes-aqueous hydrogen peroxide solution (6%, by weight). A final pH of 9.5 was obtained.

Each mixture obtained was applied to locks of grey hair containing 90% white hairs. After an action time of 30 minutes, the locks were rinsed, washed with a standard shampoo, rinsed again and then dried.

The dyeing results below were obtained.

| Examples | 1 | 2 | 3 | 4 |
|---|---|---|---|---|
| Shade | Dark blue | Dark blue-violet | Red-violet | Grey-violet |
| observed | | | | |

Examples of Dyeing in Neutral Medium

The following dye compositions were prepared:

| Examples | 5 | 6 | 7 | 8 | 9 |
|---|---|---|---|---|---|
| 4-[3-(3- | 10⁻³ mol | 10⁻³ mol | 10⁻³ mol | 10⁻³ mol | 10⁻³ mol |
| Trimethyl- | | | | | |
| silanyl- | | | | | |
| propoxy)-1- | | | | | |
| pyrrolidyl] | | | | | |
| phenylamine | | | | | |
| (base) | | | | | |
| 2-(2,4- | 10⁻³ mol | — | — | — | — |
| Diamino- | | | | | |
| phenoxy) | | | | | |
| ethanol | | | | | |
| dihydro- | | | | | |
| chloride | | | | | |
| (coupler) | | | | | |
| 3-Amino-2- | — | 10⁻³ mol | — | — | — |
| chloro-6- | | | | | |
| methyl- | | | | | |
| phenol | | | | | |
| hydro- | | | | | |
| chloride | | | | | |
| (coupler) | | | | | |
| 2-Methyl- | — | — | 10⁻³ mol | — | — |
| 5-amino- | | | | | |
| phenol | | | | | |
| (coupler) | | | | | |
| 2-Amino- | — | — | — | 10⁻³ mol | — |
| 3-pyridinol | | | | | |
| (coupler) | | | | | |
| 6-Hydroxy- | — | — | — | — | 10⁻³ mol |
| 1H-indole | | | | | |
| (coupler) | | | | | |
| Dye support | (*) | (*) | (*) | (*) | (*) |
| (2) | | | | | |
| Demineral- | 100 g | 100 g | 100 g | 100 g | 100 g |
| ized water qs | | | | | |

(*) Dye support (2) pH 7

| | |
|---|---|
| 96° ethyl alcohol | 20.8 g |
| Sodium metabisulphite as an aqueous 35% | 0.23 g A.M. |
| solution | |
| Pentasodium salt of diethylenetriaminepenta- | 0.48 g A.M. |
| acetic acid | |
| C₈-C₁₀ alkyl polyglucoside | 3.6 g A.M. |
| as an aqueous 60% solution | |
| Benzyl alcohol | 2.0 g |
| Polyethylene glycol containing 8 units of | 3.0 g |
| ethylene oxide | |
| Na₂HPO₄ | 0.28 g |
| KH₂PO₄ | 0.46 g |

At the time of use, each composition was mixed with an equal weight of 20-volumes aqueous hydrogen peroxide solution (6% by weight). A final pH of 7 was obtained.

Each mixture obtained was applied to locks of grey hair containing 90% white hairs. After an action time of 30 minutes, the locks were rinsed, washed with a standard shampoo, rinsed again and then dried.

The following dyeing results were obtained.

| Examples | 5 | 6 | 7 | 8 | 9 |
|---|---|---|---|---|---|
| Shade | Dark violet- | Dark blue- | Violet- | Violet- | Violet- |
| observed | blue | violet | grey | grey | grey |

What is claimed is:

1. A composition for dyeing keratin fibers, comprising, in a medium suitable for dyeing, at least one oxidation base chosen from para-phenylenediamine derivatives substituted with a pyrrolidyl group of formula (I), and the addition salts thereof: 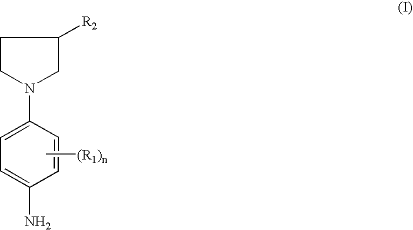
wherein
n is chosen from 0 to 4, provided that when n is greater than or equal to 2, then the radicals R₁ may be identical or different,
R₁ is chosen from halogen atoms and saturated and unsaturated, aliphatic and alicyclic C₁-C₆ hydrocarbon-based chains, wherein at least one carbon atom of the hydrocarbon-based chains may be replaced with at least one entity chosen from oxygen, nitrogen, silicon and sulphur atoms and an SO₂ group, and wherein at least one of the hydrocarbon-based chains may be substituted with at least one entity chosen from halogen atoms and hydroxyl, amino and mono- and di(C₁-C₄)alkylamino radicals; provided that the radical R₁ does not comprise a peroxide bond or a radical chosen from diazo, nitro and nitroso radicals,
R₂ is chosen from
—SiR₃R₄R₅ radicals,
linear and branched C₁-C₈ alkyl radicals, which may be unsaturated, substituted with at least one radical chosen from —SiR₃R₄R₅ radicals; wherein at least one carbon atom of the alkyl radicals may be replaced with at least one atom chosen from oxygen and nitrogen atoms and at least one of the alkyl radicals may be substituted with at least one group chosen from hydroxyl, amino, (C₁-C₆)alkylamino and di(C₁-C₆)alkylamino groups, and
triarylsilanyl, triarylsilanylalkyl, triarylsilanylalkoxy, triarylsilanylalkylamine and bis(triarylsilanylalkyl)amine radicals,
wherein R₃, R₄ and R₅, which may be identical or different, are each chosen from tri(C₁-C₄)alkylsilyl radicals; a triphenylsilyl radical; a phenyl radical; and C₁-C₆ alkyl radicals which may be substituted with at least one group chosen from tri(C₁-C₆)alkylsilyl, hydroxyl, amino, (C₁-C₆)alkylamino and di(C₁-C₆)alkylamino groups.

2. The composition according to claim 1, wherein n is equal to 0 or 1.

3. The composition according to claim 1, wherein R₁ is chosen from halogen atoms; C₁-C₄ alkyl radicals, C₁-C₄ hydroxyalkyl radicals, C₁-C₄ aminoalkyl radicals, C₁-C₄ alkoxy radicals and C₁-C₄ hydroxyalkoxy radicals.

4. The composition according to claim 3, wherein R, is chosen from methyl, isopropyl, hydroxymethyl, 2-hydroxyethyl, 1,2-dihydroxyethyl, methoxy, isopropyloxy and 2-hydroxyethoxy radicals.

5. The composition according to claim 1, wherein R₂ is chosen from trialkylsilanyl, trialkylsilanylalkyl, trialkylsilanylalkoxy, trialkylsilanylalkylamine, bis(trialkylsilanylalkyl)amine, triarylsilanyl, triarylsilanylalkyl, triarylsilanylalkoxy, triarylsilanylalkylamine and bis(triarylsilanylalkyl)amine radicals.

6. The composition according to ° claim 5, wherein R₂ is chosen from trialkylsilanyl, trialkylsilanylalkyl, trialkylsilanylalkoxy, trialkylsilanylalkylamine and bis(trialkylsilanylalkyl)amine radicals.

7. The composition according to claim 1, wherein R₃, R₄ and R₅, which may be identical or different, are each chosen from methyl, ethyl, propyl, isopropyl, butyl, isobutyl, tert-butyl, pentyl, hexyl, cyclohexyl, phenyl and tolyl radicals.

8. The composition according to claim 1, wherein the para-phenylenediamine derivatives of formula (I) are chosen from
4-[3-(3-trimethylsilanylpropoxy)-1-pyrrolidyl]phenylamine,
4-(3-trimethylsilanyl-1-pyrrolidyl)phenylamine,
4-(3-(trimethylsilanyl methoxy-pyrrolidyl)phenylamine,
[1-(4-aminophenyl)-3-pyrrolidyl]trimethylsilanylmethylamine,
4-(3-trimethylsilanylmethyl-1-pyrrolidyl)phenylamine,
[1-(4-aminophenyl)-3-pyrrolidyl]bistrimethylsilanylmethylamine,
2-(2-tri methylsilanylethyl)-4-[3-(3-trimethylsilanyl propoxy)-1-pyrrolidyl]phenylamine,
4-[3-(3-trimethylsilanylethyloxy)-1-pyrrolidyl]phenylamine, and
4-[3-(3-triphenylsilanylpropoxy)-1-pyrrolidyl]phenylamine.

9. The composition according to claim 1, wherein the at least one oxidation base is chosen from enantiomers and diasteromers of the para-phenylenediamine derivatives substituted with a pyrrolidyl group of formula (I), and the addition salts thereof.

10. The composition according to claim 1, wherein the at least one oxidation base is present in an amount ranging from 0.001% to 10% by weight relative to the total weight of the dye composition.

11. The composition according to claim 1, wherein the keratin fibers are human keratin fibers.

12. The composition according to claim 11, wherein the human keratin fibers are hair.

13. The composition according to claim 1, further comprising at least one coupler chosen from meta-phenylenediamines, meta-aminophenols, meta-diphenols, naphthalene-based couplers and heterocyclic couplers, and the addition salts thereof.

14. The composition according to claim 13, wherein the at least one coupler is present in an amount ranging from 0.001% to 10% by weight relative to the total weight of the dye composition.

15. The composition according to claim 1, further comprising at least one additional oxidation base other than the oxidation base of formula (I), wherein-the at least one additional oxidation base is chosen from para-phenylenediamines, bis(phenyl)alkylenediamines, para-aminophenols, ortho-aminophenols and heterocyclic bases, and the addition salts thereof.

16. The composition according to claim 15, wherein the at least-one additional oxidation base is present in an amount ranging from 0.001% to 10% by weight relative to the total weight of the dye composition.

17. The composition according to claim 1, further comprising at least one oxidizing agent.

18. The composition according to claim 17, wherein the at least one oxidizing agent is chosen from hydrogen peroxide, urea peroxide, alkali metal bromates, persalts, peracids and oxidase enzymes.

19. The composition according to claim 1, further comprising at least one direct dye.

20. The composition according to claim 1, wherein the medium suitable for dyeing comprises water or a mixture of water and at least one organic solvent.

21. The composition according to claim 1, further comprising at least one adjuvant chosen from anionic, cationic, nonionic, amphoteric and zwitterionic surfactants, anionic, cationic, nonionic, amphoteric and zwitterionic polymers, inorganic and organic thickeners, antioxidants, penetration agents, sequestering agents, fragrances, buffers, dispersing agents, conditioners, film-forming agents, ceramides, preserving agents, and opacifiers.

22. A process for oxidation dyeing of keratin fibers, comprising applying to the keratin fibers a dye composition in the presence of at least one oxidizing agent, for a time that is sufficient to develop a desired coloration, wherein the dye composition comprises, in a medium suitable for dyeing, at least one oxidation base chosen from para-phenylenediamine derivatives substituted with a pyrrolidyl group of formula (I), and the addition salts thereof 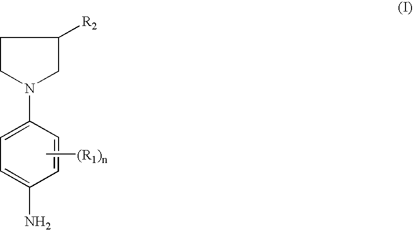
wherein
n is, chosen from 0 to 4, provided that when n is greater than or equal to 2, then the radicals R₁ may be identical or different,
R₁ is chosen from halogen atoms and saturated and unsaturated, aliphatic and alicyclic C₁-C₆ hydrocarbon-based chains, wherein at least one carbon atom of the hydrocarbon-based chains may be replaced with at least one entity chosen from oxygen, nitrogen, silicon and sulphur atoms and an SO₂ group and at least one of the hydrocarbon-based chains may be substituted with at least one entity chosen from halogen atoms and hydroxyl, amino-and mono- and di(C₁-C₄)alkylamino radicals; provided that the radical R₁ does not comprise a peroxide bond or a radical chosen from diazo, nitro and nitroso radicals,
R₂ is chosen from
—SiR₃R₄R₅ radicals,
linear and branched C₁-C₈ alkyl radicals, which may be unsaturated, substituted with at least-one radical chosen from —SiR₃R₄R₅ radicals; wherein at least one carbon atom of the alkyl radicals may be replaced with at least one atom chosen from oxygen and nitrogen atoms and at least one of the alkyl radicals may be substituted with at least one group chosen from hydroxyl, amino, (C₁-C₆)alkylamino and di(C₁-C₆)alkylamino groups, and
triarylsilanyl, triarylsilanylalkyl, triarylsilanylalkoxy, triarylsilanylalkylamine and bis(triarylsilanylalkyl)amine radicals,
wherein R₃, R₄ and R₅, which may be identical or different, are each chosen from tri(C₁-C₄)alkylsilyl radicals; a triphenylsilyl radical; a phenyl radical; and C1-C6 alkyl radicals which may be substituted with at least one-group chosen from tri(C₁-C₆)alkylsilyl, hydroxyl, amino, (C₁-C₆)alkylamino and di(C₁-C₆)alkylamino groups.

23. The process according to claim 22, wherein the at least one oxidizing agent is chosen from hydrogen peroxide, urea peroxide, alkali metal bromates, persalts, peracids and oxidase enzymes.

24. A multi-compartment device, comprising
a first compartment comprising a dye composition comprising, in a medium suitable for dyeing, at least one oxidation base chosen from para-phenylenediamine derivatives substituted with a pyrrolidyl group of formula (I), and the addition salts thereof 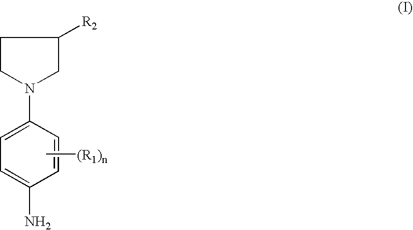
wherein
n is chosen from 0 to 4, provided that when n is greater than or equal to 2, then the radicals R₁ may be identical or different,
R₁ is chosen from halogen atoms and saturated and unsaturated, aliphatic and alicyclic C₁-C₆ hydrocarbon-based chains, wherein at least one carbon atom of the hydrocarbon-based chains may be replaced with at least one entity chosen from oxygen, nitrogen, silicon and sulphur atoms and an SO₂ group and at least one of the hydrocarbon-based chains may be substituted with at least one entity chosen from halogen atoms and hydroxyl, amino and mono- and di(C₁-C₄)alkylamino radicals; provided that the radical R₁ does not comprise a peroxide bond or a radical chosen from diazo, nitro and nitroso radicals,
R₂ is chosen from
—SiR₃R₄R₅ radicals,
linear and branched C₁-C₈ alkyl radicals, which may be unsaturated, substituted with at least one radical chosen from —SiR₃R₄R₅ radicals; wherein at least one carbon atom of the alkyl radicals may be replaced with at least one atom chosen from oxygen and nitrogen atoms and at least one of the alkyl radicals mm be substituted with at least one group chosen from hydroxyl, amino, (C₁-C₆)alkylamino and di(C₁-C₆)alkylamino groups, and
triarylsilanyl, triarylsilanylalkyl, triarylsilanylalkoxy, triarylsilanylalkylamine and bis(triarylsilanylalkyl)amine radicals,
wherein R₃, R₄ and R₅, which may be identical or different, are each chosen from tri(C₁-C₄)alkylsilyl radicals; a triphenylsilyl radical; a phenyl radical; and C₁-C₆ alkyl radicals which may be substituted with at least one group chosen from tri(C₁-C₆)alkylsilyl, hydroxyl, amino, (C₁-C₆)alkylamino and di(C₁-C₆)alkylamino groups, and
a second compartment comprising at least one oxidizing agent.

25. A process for dying keratin fibers, comprising applying to the keratin fibers at least one oxidation base chosen from para-phenylenediamine derivatives substituted with a pyrrolidyl group of formula (I), and the addition salts thereof 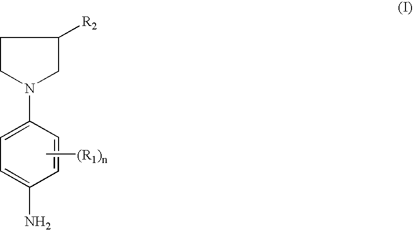
wherein
n is chosen from 0 to 4, provided that when n is greater than or equal to 2, then the radicals R₁ may be identical or different,
R₁ is chosen from halogen atoms and saturated and unsaturated, aliphatic and alicyclic C₁-C₆ hydrocarbon-based chains, wherein at least one carbon atom of the hydrocarbon-based chains may be replaced with at least one entity chosen from oxygen, nitrogen, silicon and sulphur atoms and an SO₂ group and at least one of the hydrocarbon-based chains may be substituted with at least one entity chosen from halogen atoms and hydroxyl, amino and mono- and di(C₁-C₄)alkylamino radicals; provided that the radical R₁ does not comprise a peroxide bond or a radical chosen from diazo, nitro and nitroso radicals,
R₂ is chosen from
—SiR₃R₄R₅ radicals,
linear and branched C₁-C₈ alkyl radicals, which may be unsaturated, substituted with at least one radical chosen from —SiR₃R₄R₅ radicals; wherein at least one carbon atom of the alkyl radicals may be replaced with at least one atom chosen from oxygen and nitrogen atoms and at least one of the alkyl radicals may be substituted with at least one group chosen from hydroxyl, amino, (C₁-C₆)alkylamino and di(C₁-C₆)alkylamino groups, and
triarylsilanyl, triarylsilanylalkyl, triarylsilanylalkoxy, triarylsilanylalkylamine and bis(triarylsilanylalkyl)amine radicals,
wherein R₃, R₄ and R₅, which may be identical or different, are each chosen from tri(C₁-C₄)alkylsilyl radicals; a triphenylsilyl radical; a phenyl radical; and C₁-C₆ alkyl radicals which may be substituted with, at least one group chosen from tri(C₁-C₆)alkylsilyl, hydroxyl, amino, (C₁-C₆)alkylamino and di(C₁-C₆)alkylamino groups.

26. A compound, chosen from para-phenylenediamine derivatives substituted with a pyrrolidyl group of formula (I), and the addition salts thereof 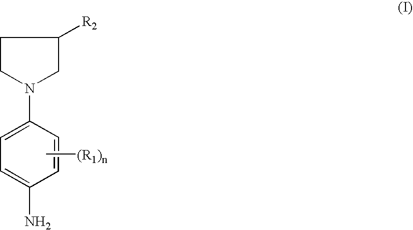
wherein
n is chosen from 0 to 4, provided that when n is greater than or equal to 2, then the radicals R₁ may be identical or different.
R₁ is chosen from halogen atoms and saturated and unsaturated, aliphatic and alicyclic C₁-C₆ hydrocarbon-based chains, wherein at least one carbon atom of the hydrocarbon-based chains may be replaced with at least one entity chosen from oxygen, nitrogen, silicon and sulphur atoms and an SO₂ group and at least one of the hydrocarbon-based chains may be substituted with at least one entity chosen from halogen atoms and hydroxyl, amino and mono- and di(C₁-C₄)alkylamino radicals; provided that the radical R₁ does not comprise a peroxide bond or a radical chosen from diazo, nitro and nitroso radicals,
R₂ is chosen from
—SiR₃R₄R₅ radicals,
linear and branched C₁-C₈ alkyl radicals, which may be unsaturated, substituted with at least one radical chosen from —SiR₃R₄R₅ radicals; wherein at least one carbon atom of the alkyl radicals may be replaced with at least one atom chosen from oxygen and nitrogen atoms and at least one of the alkyl radicals may be substituted with at least one group chosen from hydroxyl, amino, (C₁-C₆)alkylamino and di(C₁-C₆)alkylamino groups, and
triarylsilanyl, triarylsilanylalkyl, triarylsilanylalkoxy, triarylsilanylalkylamine and bis(triarylsilanylalkyl)amine radicals,
wherein R₃, R₄ and R₅, which may be identical or different, are each chosen from tri(C₁-C₄)alkylsilyl radicals; a triphenylsilyl radical; a phenyl radical; and C₁-C₆ alkyl radicals which may be substituted with at least one group chosen from tri(C₁-C₆)alkylsilyl, hydroxyl, amino, (C₁-C₆)alkylamino, and di(C₁-C₆)alkylamino groups, with the exception of 5-amino-2-((3R)-3-t-butyldimethylsilyloxy-1-pyrrolidyl)fluorobenzene.
